(19) United States
(12) Patent Application Publication
(10) Pub. No.: US 2026/0083594 A1
(43) Pub. Date: Mar. 26, 2026

(54) OCULAR DEVICE HAVING PASSTHROUGH LENS AND ELECTRONIC PROJECTION

(71) Applicant: GLAIVE MEDICAL OPTICS, INC., Carlisle, MA (US)

(72) Inventors: Wayne H. WOODS, JR., Carlisle, MA (US); Alexander J. MELVILLE, Chelmsford, MA (US); Christopher SHELBY, Shreveport, LA (US)

(21) Appl. No.: 19/341,705

(22) Filed: Sep. 26, 2025

Related U.S. Application Data
(60) Provisional application No. 63/699,256, filed on Sep. 26, 2024.
(60) Provisional application No. 63/699,293, filed on Sep. 26, 2024.
(60) Provisional application No. 63/699,295, filed on Sep. 26, 2024.

(51) Int. Cl. A61F 9/08

(52) U.S. Cl. CPC A61F 9/08

(57) ABSTRACT

An electronic ocular device is configured to be implanted in an eye and includes: a passthrough aperture defining an optical path extending through the electronic device from a front side of the electronic device to a back side of the electronic device; multiple imaging systems configured to receive incoming light at the eye; and multiple projection systems configured to project images onto multiple different regions of a retina of the eye based on the incoming light received by the imaging systems. The optical path through the passthrough aperture may be selectively blocked or unblocked such that electronic device can be selectively adjusted between a first mode of vision and a second mode of vision. Power used by the electronic ocular device is minimized by operating different ones of the projection systems at different resolutions based on different eye resolution at different regions of the retina.

### RELATED APPLICATIONS

The application claims priority to U.S. provisional patent application No. 63/699,256, filed Sep. 26, 2024, U.S. provisional patent application No. 63/699,293, filed Sep. 26, 2024, and U.S. provisional patent application No. 63/699,295, filed Sep. 26, 2024, the contents of each of which are incorporated by reference herein in their entirety.

### BACKGROUND

The present disclosure relates to electronic vision systems. Such systems have a variety of application such as therapeutic (e.g., to treat vision disorders such as blindness), professional, and recreation.

### SUMMARY

In accordance with aspects of the present disclosure, an electronic device is configured to be implanted in the eye of a user to enhance or artificially provide vision in the eye. In various embodiments, the electronic device includes a passthrough aperture defining an optical path extending through the electronic device from a front side of the electronic device to a back side of the electronic device, multiple electronic imaging systems configured to receive incoming light at the eye, and multiple electronic projection systems configured to project images onto multiple different regions of a retina of the eye based on the incoming light received by the imaging systems. In accordance with aspects of the present disclosure, the electronic device includes a system that can selectively block or unblock the optical path through the passthrough aperture. In this manner, the electronic device can be selectively adjusted between: a first mode of vision in which the passthrough aperture is unblocked and real-world images seen through the passthrough aperture are augmented using images projected onto the retina by the electronic projection systems; a second mode of vision in which the passthrough aperture is blocked and images are projected onto the retina by the electronic projection systems; and a third mode in which vision is provided only through the passthrough aperture and in which the electronic projection systems do not project images onto the retina.

In accordance with aspects of the present disclosure, the images that are projected onto the retina by the electronic projection systems may correspond to real world images received at the electronic imaging systems to provide a baseline of standard vision to the user. In various embodiments, the projected images may be enhanced using one or more of selective magnification of areas or objects in the images, thermal imaging, brightness and contrast control, color saturation/desaturation, or image sharpening. Theses enhancements may be achieved using one or more image processing techniques carried out by control circuitry in the electronic device or by an external computer device that communicates wirelessly with the electronic device.

In accordance with some aspects of the present disclosure, the electronic device is configured to function as a virtual reality (VR) device in the second mode of vision in which the passthrough aperture is blocked and images are projected onto the retina by the electronic projection systems. In a VR mode, the electronic device may communicate with an external computer device for generating the images that are projected onto the retina by the electronic projection systems.

In accordance with aspects of the present disclosure, the electronic device is configured to function as an augmented reality (AR) device in the first or second modes of vision described above. In one example of an AR mode of the electronic device, real-world images seen through the passthrough aperture are augmented using images projected onto the retina by the electronic projection systems. In another example of an AR mode of the electronic device, the optical path through the passthrough aperture may be blocked, and real-world images captured by the electronic imaging systems may be augmented electronically by the electronic device or an external computer device, and these augmented images may be projected onto the retina by the electronic projection systems. In embodiments, such augmentation may include but is not limited to adding closed-captioning, adding descriptive text, adding user-defined visual information, and other information specific to the incoming visual information (e.g., real world images) received from the front-facing electronic imaging systems.

In accordance with some aspects of the present disclosure, the electronic device is controlled by the user using voice control, predefined eye movements, predefined face movements, or predefined hand movements. In embodiments, this enables the user to choose between the various display modes described above, e.g., standard vision, enhanced vision, virtual reality, or augmented reality.

In accordance with some aspects of the present disclosure, the electronic device is configured to simultaneously project frontal vision images onto a first region of the retina at a relatively high resolution and peripheral vision images onto a second region of the retina at relatively low resolution. In various implementations, the electronic device includes a first electronic imaging system and a first electronic projection system that are configured to receive incoming light associated with frontal vision and project images corresponding to this incoming light at the frontal vision region of the retina. In various implementations, the ocular device additionally includes a second electronic imaging system and a second electronic projection system that are configured to receive incoming light associated with peripheral vision and project images corresponding to this incoming light at the peripheral vision region of the retina. Because the eye detects frontal vision and peripheral vision at different resolutions, and because the frontal vision region of the retina is a different size than the peripheral vision region of the retina, embodiments of the present disclosure are optimized to minimize the number of pixels used in the respective projection systems when simultaneously projecting frontal vision and peripheral vision images onto the retina. Minimizing the number of pixels used, while simultaneously providing adequate frontal and peripheral vision, enables reducing the physical size of the electronic device and reducing the electrical power consumed by the electronic device, both of which are desirable in an electronic ocular implant. In this manner, implementations according to the present disclosure may be used for professional purposes, recreational purposes, or to treat blindness from a multitude of disparate sources can be treated, including eye injuries, retinal diseases, and age-related degeneration.

In an aspect of the present disclosure, there is an electronic device configured to be implanted in or on an eye, the electronic device comprising: a passthrough aperture defining an optical path extending through the electronic device from a front side of the electronic device to a back side of the electronic device; multiple imaging systems configured to receive incoming light at the eye; and multiple projection systems configured to project images onto multiple different regions of a retina of the eye based on the incoming light received by the imaging systems.

In various embodiments of the electronic device, the different regions of the retina include: a first region associated with frontal vision; and a second region associated with peripheral vision.

In various embodiments of the electronic device, the projection systems comprise: a first projection system configured to project first images onto the first region at a first resolution; and a second projection system configured to project second images onto the second region at a second resolution that is lower than the first resolution.

In various embodiments of the electronic device, the projection systems are configured to simultaneously project respective different images onto respective ones of the different regions of the retina.

In various embodiments of the electronic device, the electronic device further comprises an electronic layer in the optical path, wherein the electronic layer is adjustable between a first state in which the electronic layer is transparent and a second state in which the electronic layer is opaque and blocks the optical path.

In various embodiments of the electronic device, the electronic device further comprises a polarized lens in the optical path.

In various embodiments of the electronic device, the electronic device further comprises an external device comprising a polarized layer having a first orientation of polarization, wherein the polarized lens has a second orientation of polarization opposite the first orientation of polarization such that the polarized layer and the polarized lens combine to block the optical path when the external device is placed in front of the electronic device.

In various embodiments of the electronic device, the polarized layer is optically transparent to the imaging systems when the external device is placed in front of the electronic device.

In various embodiments of the electronic device, the electronic device comprises a central body and haptics.

In various embodiments of the electronic device, the passthrough aperture, the imaging systems, and the projection systems are contained in a chip or chip stack, or attached to a printed circuit board, in the central body.

In various embodiments of the electronic device, the electronic device further comprises control circuitry, a power source, a wireless communication system, and a wireless charging system.

In various embodiments of the electronic device, the electronic device further comprises a structure extending between the central body and haptics.

In various embodiments of the electronic device, the structure, the central body, and the haptics define a seat that is configured to receive an iris of the eye when the electronic device is implanted in the eye.

In various embodiments of the electronic device, the electronic device is implanted in the eye the haptics are configured to be in an anterior chamber of the eye forward of the iris and the central body is configured to be behind the iris.

In accordance with aspects of the present disclosure, a method comprising implanting various embodiments of the electronic device in the eye of a user such that the haptics are in the anterior chamber of the eye forward of the iris and the central body is behind the iris.

In various embodiments of the electronic device, the structure comprises one or more walls that define an opening and the passthrough aperture and the imaging systems are arranged within a perimeter of the opening when viewed from a front of the electronic device.

In various embodiments of the electronic device, the central body comprises a case having that encapsulates a chip or chip stack containing the passthrough aperture, the imaging systems, and the projection systems, a first end of the structure is connected to the case, and the haptics are connected to a second end of the structure opposite the first end of the structure.

In various embodiments of the electronic device, the electronic device configured to provide vision enhancement comprising one of selective magnification of areas or objects, thermal imaging, brightness and contrast control, color saturation/desaturation, or image sharpening.

In various embodiments of the electronic device, the electronic device is configured to operate as a virtual reality device.

In various embodiments of the electronic device, the electronic device is configured to operate as an augmented reality device.

In various embodiments of the electronic device, the electronic device is configured to be controlled using voice control, predefined eye movements, predefined face movements, or predefined hand movements.

In various embodiments of the electronic device, the electronic device comprises one of: a contact lens that is configured to be placed on an exterior of the eye; or an implantable contact lens that is configured to be implanted in the eye.

### BRIEF DESCRIPTION OF THE DRAWINGS

Various features and advantages of the technology of the present disclosure will be apparent from the following description of particular examples of those technologies, and as illustrated in the accompanying drawings. The drawings are not necessarily to scale; the emphasis instead is placed on illustrating the principles of the technological concepts. In the drawings, like reference characters may refer to the same parts throughout the different views. The drawings depict only illustrative examples of the present disclosure and are not limiting in scope.

FIG. 1 shows a diagram of an eye with an implanted ocular device in accordance with aspects of the present disclosure.

FIG. 2 diagrammatically shows a first region and a second region on a portion of a retina in accordance with aspects of the present disclosure.

FIG. 3 shows diagrammatic cross-sectional views of components of the ocular device of FIG. 1 in accordance with aspects of the present disclosure.

FIG. 4 diagrammatically shows a front view of an exemplary implementation of the ocular device of FIG. 1 in accordance with aspects of the present disclosure.

FIG. 5 shows an exemplary implementation of haptics and a pupil collar of an ocular device in accordance with aspects of the present disclosure.

FIG. 6 shows an example of an ocular device implanted in an eye in accordance with aspects of the present disclosure.

FIG. 7 illustrates wireless charging in accordance with aspects of the present disclosure.

FIG. 8 diagrammatically shows an exemplary optimization of relative locations of the passthrough aperture and the imaging systems in accordance with aspects of the present disclosure.

FIG. 9 shows a plot of eye resolution versus off-axis angle in accordance with aspects of the present disclosure.

### DETAILED DESCRIPTION

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, the description taken with the drawings making apparent to those skilled in the art how the several forms of the present disclosure may be embodied in practice.

Various embodiment of the present disclosure include an implantable electronic ocular device that utilizes multiple imaging systems with multiple projection systems to simultaneously project frontal vision images and peripheral vison images onto a retina while to minimizing power and pixels used to perform such image projections. Implementations achieve this minimizing by making use of natural resolution versus angle profile of the human retina. The human retina has a very high resolution (“20/20” vision) of about 60 pixels/degree, but this high resolution is only in a very narrow angular range on the retina: roughly +/−1.5 degrees from the center of the retina. However, the vast majority of the retinal tissue has a much lower resolution. For example, the areas of the retina nearer to 50 to 60 degrees off center is down around 2 pixels per degree. This makes intuitive sense as we know that the peripheral vision has far less resolution than the frontal vision. For a projection system concerned with augmented reality, this is not an issue: such systems focus their projections primarily to overlay data over the frontal vision of the user. However, for applications that are trying to provide or augment the peripheral vision of the user, peripheral projection is of particular importance. Given that it is important to project to the frontal vision and the peripheral vision, one does not have great choices in how to do so. For example, if a single lens/screen projection system is used, then one must project at 60 pixels/degree to allow the maximum forward vision resolution. To project that resolution across +/−60 degrees would require 120 degrees×60 pixels/degree or 7200 pixels which would require 7200 pixels in X and Y which is 7200×7200 pixels or over 50 million pixels. If, alternatively, one used a single lens/screen system to only project at the low-resolution level of the far-peripheral area of the retina, then this would require 2 pixels/degree×120 degrees or 240 pixels, leading to 240×240 pixels or 57,600 pixels. However, if projecting only in low resolution, then the forward vision no longer has any high-resolution vision. Both of these solutions are undesirable because in the first scenario there is way more power/pixels being used that cannot even be resolved in the peripheral areas, and in the second case there is no longer any high-resolution imaging possible.

Implementations according to the present disclosure provide a solution to this problem by using multiple projection systems to simultaneously project images to different areas of the eye. The relatively small area of the retina that is associated with high-resolution frontal vision is projected to with a high-resolution projection system, while the relatively large area of the retina that is associated with low-resolution peripheral vision is projected to with a low-resolution projection system. In this manner, the overall number of pixels included in, and powered by, the ocular implant can be greatly reduced while simultaneously providing perfectly adequate frontal and peripheral vision to the user.

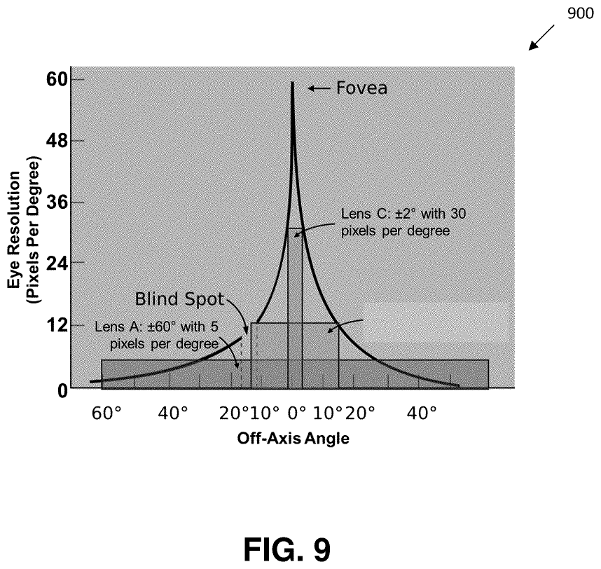

FIG. 9 shows a plot 900 of eye resolution versus off-axis angle in accordance with aspects of the present disclosure. In FIG. 9, the curved line represents resolution of the eye plotted against the off-axis angle, which is the angle from the pupil between the optical axis of the eye and the retina. Embodiments of the present disclosure include an ocular device having different projection systems with different lenses designed to minimize the number of pixels while maximizing the user's restored vision, represented in FIG. 9 as lenses A and C, providing peripheral vision and central vision, respectively

An ocular device in accordance with aspects of the present disclosure and having different projection systems with lenses A and C as shown in FIG. 9 provides using far less pixels than 50 million pixels as would be the case with a single resolution system. For example, in an ocular device according to FIG. 9, a first projection system having lens C projects relatively high resolution (20/40 vision) or 30 pixels/degree at +/−2 degrees off-axis, which is a total of 4 degrees×30 pixels/degree leading to a need of 120×120=14,400 pixels for this first region. In this example of an ocular device according to FIG. 9, a second projection system having lens A projects a relatively low resolution of 5 pixels per degree over +/−60 degrees leading to 720×720 pixels or 518,500 pixels in this third region. This example of an ocular device according to FIG. 9, having two different systems projecting images at two different resolutions, covers the entire retinal area with an approximation of the eye's natural resolution profile. This two system approach utilizes only 532,900 pixels as opposed to 51,840,000 pixels for a single lens that can cover the whole retinal area and allow high-resolution forward vision. This means that for about 1% of the pixels and power, the eye can be provided with a good approximation of a visual field. Moreover, as described herein, this pixel count (i.e., 532,900 pixels) can be further reduced because the middle pixels low resolution system (i.e., pixels correspond to areas where projection regions overlap) are not needed and can be selectively turned off to avoid image overlap and further reduce power consumption of the ocular device. Various embodiments utilize this same reasoning on the imaging (e.g., camera) side of the ocular device and, thus, may utilize imaging systems having different resolutions to reduce pixel count on the image sensors, since there is there is little point in taking in more data that the eye can resolve. In this manner, implementations having multiple imaging systems and multiple projection systems, each tailored to different resolutions based on differing eye resolutions, drastically reduces pixel count and power required in the ocular device.

In the technical field of electronic ocular implants, embodiments of the present disclosure provide the advantage of minimized power because using less pixels results in less power consumed by the projection systems (e.g., LED power), using less pixels results in less power consumed by the imaging systems (e.g., CMOS imager power), and using less pixels results in less internal data transfer (about 1% of what would otherwise be needed) between the imaging and projection systems which means much less bandwidth and data transfer power required.

Some embodiments use multiple different imaging systems each having a respective image sensor (e.g., CMOS image sensor) and lens, and multiple different projection systems each having a respective light projecting device (e.g., LED panel) and lens. In these embodiments, power savings may be achieved by using fewer pixels in the image sensor of the imaging system and the light generation device of the projection system used for projecting peripheral vision. As described herein, peripheral vision regions of the eye cannot resolve the same resolution as frontal vision regions, and implementations leverage this fact to reduce the number of pixels utilized in the peripheral vison systems compared to the frontal vision systems. Using different number of pixels in the two different systems as described herein can provide almost a 99% reduction in total number of pixels in the device compared to a device that projects high resolution over the entire +/−60 degrees region of the retina.

Some embodiments used a single shared image sensor for the multiple imaging systems (e.g., multiple different lenses arranged over different sections of a single CMOS image sensor) and a shared light projecting device for the multiple projection systems (e.g., multiple different lenses arranged over different sections of a single LED panel). In these embodiments, power savings can be achieved by selectively turning off varying amounts of pixels in the different sections of the LED panel based on the different resolutions of projection associated with the different sections of the LED panel. These embodiment also benefit from ease of manufacturing since only a single CMOS image sensor and a single LED panel are used.

Implementations in accordance with aspects of the present disclosure thus may be used to provide an electronic ocular implant including a multiple lens/periscope/prism system on the projection side and a multiple lens/periscope/prism system on image side. Respective ones of the lens/periscope/prism systems may be used to provide linear path projection or circuitous path projection as described herein. The electronic ocular implant may be used to perform a method of optimally projection images onto the frontal and peripheral regiones of the retina. As described herein, the electronic ocular implant may also be used to perform a method of optimally imaging the retina of the eye in which the ocular device is implanted.

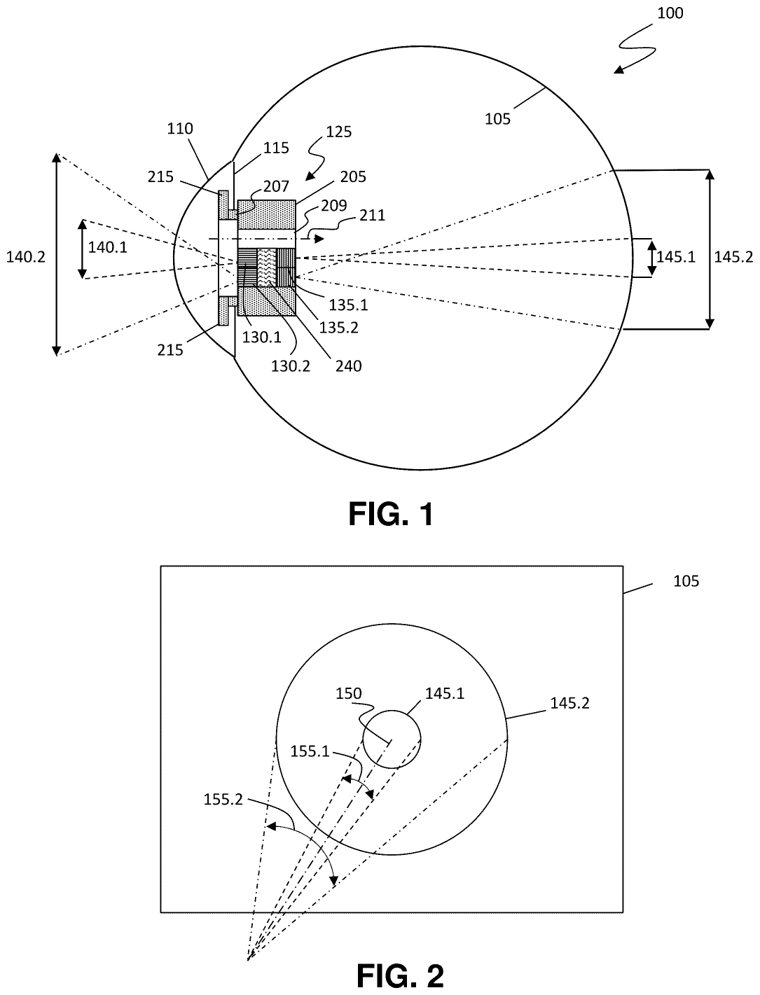

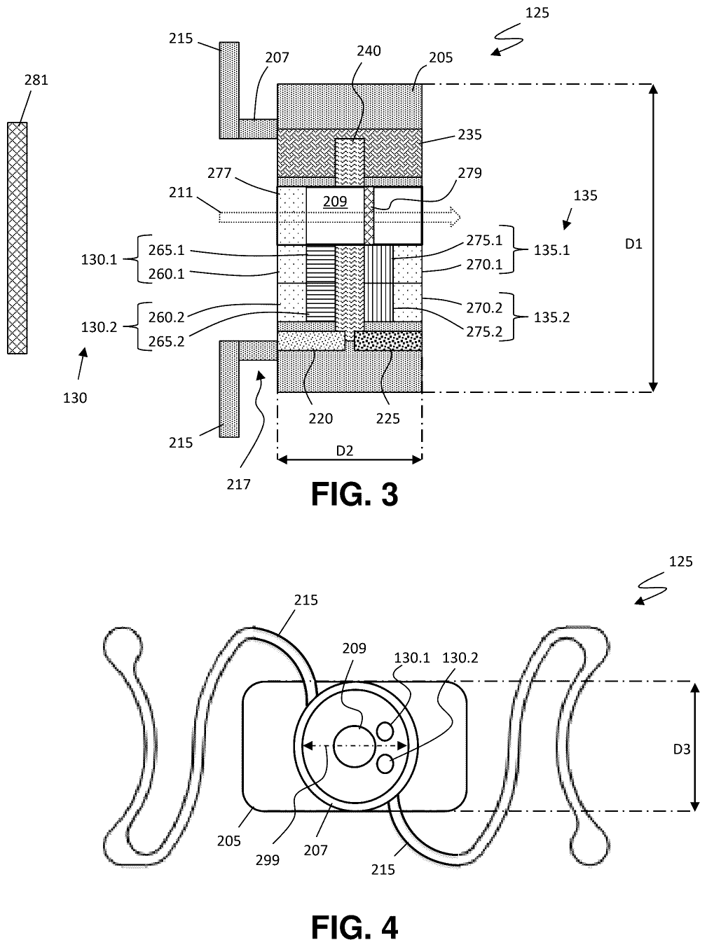

FIG. 1 shows a diagram of an eye 100 with an implanted ocular device 125 in accordance with aspects of the present disclosure. The eye 100 includes a retina 105 at a posterior region and a cornea 110 and iris 115 at an anterior region. The ocular device 125 is also referred to herein as an electronic device. FIG. 3 shows diagrammatic cross-sectional views of components of the ocular device of FIG. 1 in accordance with aspects of the present disclosure.

With reference to FIGS. 1 and 3, the ocular device 125 is configured to be implanted in the eye 100 at the anterior region, e.g., with haptics 215 of the ocular device 125 in an anterior chamber of the eye forward of the iris 115 and a central body 205 of the ocular device 125 behind the iris 115. In various embodiments, the ocular device 125 comprises: a passthrough aperture 209 defining an optical path 211 extending through the ocular device 125 from a front side of the ocular device 125 to a back side of the ocular device 125; multiple imaging systems 130 configured to receive incoming light at the eye 100; and multiple projection systems 135 configured to project images onto multiple different regions of a retina 105 of the eye based on the incoming light received by the imaging systems 130.

In various embodiments, and as shown in FIGS. 1 and 3, the ocular device 125 comprises a central body 205 and haptics 215. In some embodiments, the passthrough aperture 209, the imaging systems, and the projection systems are contained in a chip or chip stack in the central body 205. As shown in FIG. 3, the ocular device 125 may further comprising control circuitry 240, a power source 235, a wireless communication system 225, and a wireless charging system 220, as described herein.

In accordance with some aspects of the present disclosure, the ocular device 125 includes a structure 207 extending between the central body 205 and the haptics 215. In embodiments, the structure 207 comprises a pupil collar and the structure 207, the central body 205, and the haptics 215 define a seat 217 that is configured to fill the pupil opening defined by the iris 115 and receive the iris 115 when the ocular device 125 device is implanted in the eye, which beneficially prevents the iris 115 from contracting the pupil to so small as shape as to otherwise obstruct light from entering the passthrough aperture 209 and the imaging systems 130.

In the example shown in FIGS. 1 and 3, the ocular device 125 includes two imaging systems 130 (i.e., a first imaging system 130.1 and a second imaging system 130.2) and two projection systems 135 (i.e., a first projection system 135.1 and a second projection system 135.2). An ocular device in accordance with aspects of the present disclosure is not limited to two imaging systems and two projection systems, and other numbers of each type of system may be used in different implementations.

In accordance with aspects of the present disclosure, the first imaging system 130.1 is associated with and operatively connected to the first projection system 135.1, the second imaging system 130.2 is associated with and operatively connected to the second projection system 135.2, and the ocular device 125 is arranged such that: the first imaging system 130.1 is configured to receive incoming light from a first field of view 140.1 outside the eye 100 and the first projection system 135.1 is configured to project light onto a first region 145.1 of the retina 105 at a first resolution based on signals generated by the first imaging system 130.1 in response to light received by the first imaging system 130.1; and the second imaging system 130.2 is configured to receive incoming light from a second field of view 140.2 outside the eye 100 and the second projection system 135.2 is configured to project light onto a second region 145.2 of the retina 105 at a second resolution (that is different than the first resolution) based on signals generated by the second imaging system 130.2 in response to light received by the second imaging system 130.2.

In accordance with aspects of the present disclosure, the first region 145.1 of the retina 105 is a region of the retina 105 that includes receptors for frontal vision, and the second region 145.2 of the retina 105 is a region of the retina that includes receptors for peripheral vision. In one example, the first region 145.1 of the retina 105 includes primarily cones and is in the fovea at the central region of the retina 105, and the second region 145.2 of the retina 105 includes primarily rods and is in the outer regions of the retina 105 surrounding the fovea. Cones are used for frontal vision and have high acuity which allows for sharp, detailed vision. Rods are used for peripheral vision and have low acuity that provides less sharp and less detailed vision than achieved with cones.

In accordance with aspects of the present disclosure, the first projection system 135.1 is configured to project light onto the first region 145.1 at a first resolution that is relatively high, and the second projection system 135.2 is configured to project light onto the second region 145.2 at a second resolution that is relatively low, where the first resolution is higher than the second resolution. In embodiments, the first resolution is relatively high in order to provide more detailed images for the user's frontal vision, and the second resolution is relatively low in order to provide less detailed images for the user's peripheral vision. In embodiments, the number of pixels used in the first projection system 135.1 is tailored to the size (e.g., area) of the first region 145.1 and the magnitude of the first resolution, and the number of pixels used in the second projection system 135.2 is tailored to the size (e.g., area) of the second region 145.2 and the magnitude of the second resolution. In this manner, each of the first projection system 135.1 and the second projection system 135.2 may be optimized to minimize its physical size and power consumption based on the respective numbers of pixels used by the respective projection systems.

FIG. 2 diagrammatically shows the first region 145.1 and the second region 145.2 on a portion of the retina 105, looking inward along an axis 150 that intersects the fovea of the eye 100. In some embodiments, the first region 145.1 and the second region 145.2 may be defined based on an off-axis angle, which as used herein refers to the angle defined between the axis 150 extending from the center of the pupil to the center of the fovea and a line extending from the center of the pupil to another point on the retina. In one example, the first region 145.1 is the area of the retina 105 defined within a first range of off-axis angles 155.1 (e.g., off-axis angles within a range of +/−2 degrees), the first resolution is 30 pixels per degree, the second region 145.2 is the area of the retina 105 defined within a second range of off-axis angle 155.2 (e.g., off-axis angles within a range of +/−60) degrees and not including the first region 145.1, and the second resolution is 5 pixels per degree. In this manner, the ocular device 125 is configured to project a relatively high-resolution image on the first region 145.1 defined by the first off-axis angle 155.1, and to project a relatively low-resolution image on the second region 145.2 defined by the second off-axis angle 155.2.

In various embodiments, the ocular device 125 stores data that is based on a mapping of the retina 105 for a particular eye of a particular user and that is used to control the operation of the first projection system 135.1 and the second projection system 135.2 so that the first projection system 135.1 projects light only onto the first region 145.1 and so that the second projection system 135.2 projects light only onto the second region 145.2. In embodiments, the second projection system 135.2 does not project light onto the first region 145.1 so as to avoid overlap of projected images in the first region 145.1. This may be accomplished, in one example, by turning off (e.g., not utilizing) light emitting pixels in the second projection system 135.2 that are mapped to the first region 145.1. In some embodiments, the data that is based on the mapping of the retina 105 is stored on an external computer device and may be wirelessly communicated to the ocular device 125.

FIG. 3 is a diagrammatic cross-sectional view of the ocular device 125 of FIG. 1 showing additional details of the ocular device 125 in accordance with aspects of the present disclosure. FIG. 4 is a diagrammatic front view (e.g., looking in the direction of arrow 211 in FIG. 3) of an exemplary implementation of the ocular device 125 of FIG. 1 in accordance with aspects of the present disclosure. The ocular device 125 is depicted diagrammatically in FIGS. 3 and 4 and is not limited to the shape and/or arrangement of components shown in FIGS. 3 and 4 unless explicitly stated herein.

In embodiments, and as shown in FIGS. 3 and 4, the ocular device 125 comprises a central body 205, haptics 215, and a structure 207 between and connecting the central body 205 and the haptics 215. In embodiments, the haptics 215 are in the form of wings or tabs that each extend outward from the central body 205. The central body 205 may be made in the form of a single piece composed of materials such as acrylic and/or silicone lens material that encapsulates the electronics the ocular device 125. In another example, the central body 205 may be made in the form of a multi-piece (e.g., two piece) case that can be opened and closed and that houses the electronics of the ocular device 125.

In embodiments, the electronics of the ocular device 125 include a wireless charging coil 220, a wireless communication antenna 225, multiple imaging systems 130 (e.g., first imaging system 130.1 and second imaging system 130.2), multiple projection systems 135 (e.g., first projection system 135.1 and second projection system 135.2), a power source 235, and control circuitry 240. In some configurations, the charging coil 220 and the wireless communication antenna 225 are embedded in one or both of the haptics 215 and the imaging systems, projection systems, power source 235, and control circuitry 240 are integrated in chip stack contained in the central body 205 of the body 205, although other arrangements may be utilized. In some embodiments, the functions of the charging coil 220 and the wireless communication antenna 225 are combined in a single coil of electrical conductor (e.g., wire). In some embodiments, the charging coil 220 and the wireless communication antenna 225 are formed in the shape of a coil wrapped around an interior of the body 205.

In accordance with aspects of the present disclosure, respective ones of the imaging systems 130 are configured to receive incoming light from outside the eye when the ocular device 125 is implanted in an eye of a user, e.g., as shown in FIGS. 1 and 6. In embodiments, each respective one of the imaging systems comprises one or more image sensors that receive incoming light from outside the eye and that provide input to the control circuitry 240 based on the received light. The image sensors may comprise any suitable type of on-chip imaging technology, such as a charge-coupled device (CCD) or a complementary metal-oxide-semiconductor (CMOS) image sensor. The respective ones of the imaging systems may comprise different regions of a single image sensor or may comprise separate image sensors. Each respective one of the imaging systems may include one or more specialized local lens structures to enhance functionality of the ocular device 125. For example, the first imaging system 130.1 may comprise a first imaging lens 260.1 and a first image sensor 265.1, the first imaging lens 260.1 having first optical characteristics (e.g., a standard-angle lens). In this example, the second imaging system 130.2 may comprise a second imaging lens 260.2 and a second image sensor 265.2, the second imaging lens 260.2 having second optical characteristics (e.g., a wide-angle lens) different than the first optical characteristics. In some embodiments, the output of each respective imaging system is a respective time-dependent electronic signal to the control circuitry 240 that corresponds to incoming light received at the respective imaging system.

In accordance with aspects of the present disclosure, respective ones of the projection systems 135 are configured to project light (e.g., images) onto respective defined regions of the retina, such as the first region 145.1 and second region 145.2 as described with respect to FIGS. 1 and 2, in one example. In embodiments, each respective one of the projection systems receives respective control signals from the control circuitry 240 and projects a respective image onto a respective region of the retina based on the control signals. The projection systems may comprise any suitable light generation devices, such as a light generation panel comprising light emitting diodes (LEDs), as but one example. The respective ones of the projection systems may comprise different regions of a single light generation device or may comprise separate light generation devices.

Each respective one of the projection systems 135 may include one or more specialized local lens structures to enhance functionality of the ocular device 125. For example, the first projection system 135.1 may comprise a first projection lens 270.1 and a first light generation device 275.1 (e.g., a first LED panel including a first number of pixels at a first pitch), the first projection lens 270.1 having first optical characteristics (e.g., a standard-angle lens). In this example, the second projection system 135.2 may comprise a second projection lens 270.2 and a second light generation device 275.2 (e.g., a second LED panel including a second number of pixels at a second pitch), the second projection lens 270.2 having second optical characteristics (e.g., a wide-angle lens) different than the first optical characteristics.

In embodiments, the projection lens used in a respective one of the projection systems 135 is optically tailored to the resolution displayed by the respective projection system and size of the region projected onto by the respective projection system. For example, a first projection lens included in the first projection system 135.1 may be manufactured with first optical characteristics (e.g., focal length, field of view, refractive index, etc.) that are specifically configured to deliver an image onto an area the size of the first region 145.1 at the first resolution used by the first projection system 135.1, and a second projection lens included in the second projection system 135.2 may be manufactured with second optical characteristics (e.g., focal length, field of view, refractive index, etc.) that are different from the first optical characteristics and that are specifically configured to deliver an image onto an area the size of the second region 145.2 at the second resolution used by the second projection system 135.2.

The ocular device 125 may be structured and arranged with one or more of the projection lenses (e.g., first projection lens 265.1 and second projection lens 265.2) to provide linear path projection as described in U.S. application Ser. No. 19/195,967, filed May 1, 2025, titled “LINEAR PATH IMAGE PROJECTION ONTO RETINA FROM ELECTRONIC INTRAOCULAR LENS (IOL)” and published as US Patent Application Publication No. [to be added after publication], the contents of which are incorporated by reference herein in their entirety. For example, one or more of the projection systems (e.g., the first projection system 135.1) may include an enclosure, lens (e.g., first projection lens 270.1), and display (e.g., first light generation device 275.1) arranged in a manner described in U.S. application Ser. No. 19/195,967.

Additionally or alternatively, the ocular device 125 may be structured and arranged with one or more of the projection lenses (e.g., first projection lens 265.1 and second projection lens 265.2) to provide circuitous path projection as described in U.S. application Ser. No. 19/195,971, filed May 1, 2025, titled “CIRCUITOUS PATH IMAGE PROJECTION ONTO RETINA FROM ELECTRONIC INTRAOCULAR LENS (IOL)” and published as US Patent Application Publication No. [to be added after publication], the contents of which are incorporated by reference herein in their entirety. For example, one or more of the projection systems (e.g., the first projection system 135.1) may include an enclosure, lens (e.g., first projection lens 270.1), and display (e.g., first light generation device 275.1) arranged in a manner described in U.S. application Ser. No. 19/195,971.

The control circuitry 240 may comprise a controller, a central processing unit (CPU), an application-specific integrated circuit (ASIC), a field-programmable gate array (FPGA), a system-on-chip (SoC), or other processing device or computing device for processing executable instructions. The electronics of the ocular device 125 may be composed of sub-circuits which may be on disparate chip materials and made with disparate technologies, such as Si, SiC, SiN, InP, GaAs, Liquid Crystal, etc. This integrated system can be stacked in as shown in FIG. 4, with the connections between circuit elements being formed using BGA/C4/micro-BGA, through substrate (or silicon) vias (TSVs), and micro-TSVs. Physical connections between layers can be through solder or oxide bonding techniques. In one example, the control circuitry 240 comprises a printed circuit board and the imaging systems 130 and projection systems 135 may comprise chips that are physically and electronically connected to the printed circuit board.

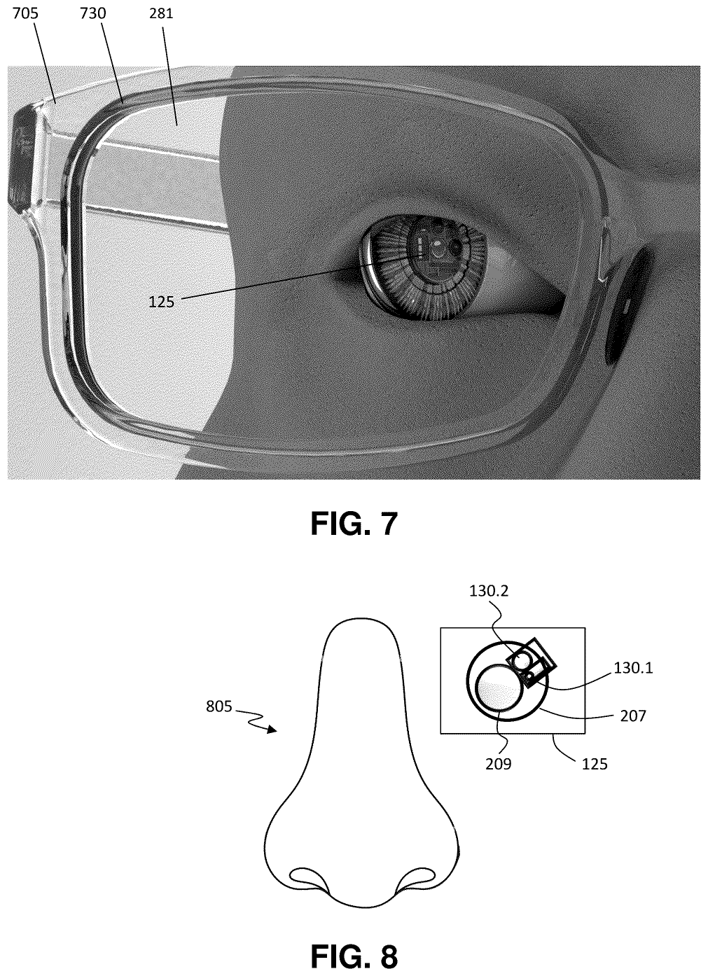

In embodiments, the wireless charging coil 220 comprises one or more inductive coupling coils and the power source 235 comprises a rechargeable battery that can be wirelessly recharged through inductive coupling using the wireless charging coil 220. In this regard, although not shown, the wireless charging coil 220 may be operatively connected to the power source 235, e.g., directly or indirectly via the control circuitry 240. The wireless charging coil 220 may be used to wirelessly charge the power source 235 using an external charging device, e.g., as shown in FIG. 7. The wireless communication antenna 225 may be in the haptics 215, in the central body 205, or in a chip or chip stack that contains the control circuitry 240, or any combination thereof. In some embodiments, the power source 235 does not store an appreciable amount of electrical power or the power source 235 is omitted altogether. In such embodiments, the ocular device 125 may be configured to use the electronics only when being wirelessly powered by an external device such as eyeglasses 705 show in FIG. 7. In these embodiments, the ocular device 125 may be configured to provide the first or second modes of vision described herein when the user is wearing the external device that wirelessly provides power to the ocular device 125, and the ocular device 125 may be configured to provide only the third mode of vision described herein when the user is not wearing the external device that wirelessly provides power to the ocular device 125. In such embodiments, the ocular device 125 may be configured to automatically turn on or turn off the electronics, respectively based on the user donning or doffing the external device that wirelessly provides power to the ocular device 125.

In accordance with aspects of this disclosure, and with continued reference to FIG. 3, the passthrough aperture 209 defining an optical path 211 extending through the ocular device 125 from a front side of the ocular device 125 to a back side of the ocular device 125. The optical pathway is shown as linear in FIG. 3 but could alternatively be nonlinear via use of reflective surfaces (e.g., similar to a periscope configuration). In various embodiments, the ocular device 125 includes a passthrough lens 277 in the passthrough aperture 209. The passthrough lens 277 may comprise a wide-angle optical lens, for example.

In accordance with aspects of the present disclosure, the ocular device 125 includes a structure 279 that is configured to selectively block or unblock the optical path 211 through the passthrough aperture 209. In one example, the structure 279 comprises an electronic layer in the optical path 211, wherein the electronic layer is adjustable between a first state in which the electronic layer is transparent and a second state in which the electronic layer is opaque and blocks the optical path. In this example, the electronic layer may comprise a light valve (e.g., an LCD light valve) that is operatively connected to the control circuitry 240 and that is selectively controlled by the control circuitry to be either transparent or opaque. Structure 279 may be at any location along the optical path 211. For example, FIG. 3 shows the structure 279 at an intermediate location inside the passthrough aperture 209; however the structure 279 may alternatively be located at the front end of the passthrough aperture 209 or the back end of the passthrough aperture 209 or any other location within the passthrough aperture 209.

In another example, the structure 279 comprises a polarized lens in the optical path 211. In this example, the polarized lens has a particular orientation of polarization. In this example, an external device comprises a polarized layer 281 that has an orientation of polarization that is opposite that of the structure 279. For example, the polarized lens of the structure 279 may have a left-hand polarization and the polarized layer 281 may have a right-hand polarization. In this manner, when the external device comprising the polarized layer 281 is located in front of the ocular device 125, the polarized layer 281 and the polarized lens of the structure 279 combine to block the optical path 211 so that the optical path 211 is essentially opaque to light from outside the eye in which the ocular device 125 is implanted. In embodiments, the polarized layer 281 is essentially optically transparent the imaging systems 130 such that the imaging systems 130 are essentially not optically blocked when the polarized layer 281 is located in front of the ocular device 125. In various embodiments, the polarized layer 281 is contained an external device such as eyeglasses, a monocle, or a contact lens. In this manner, a user may selectively block and unblock the optical path 211 by putting on and taking off the external device containing the polarized layer 281.

In accordance with aspects of the present disclosure, the structure 279 enables the ocular device 125 to be selectively adjusted between: a first mode of vision in which the passthrough aperture 209 is unblocked and real-world images seen through the passthrough aperture 209 are augmented using images projected onto the retina by the electronic projection systems 135; a second mode of vision in which the passthrough aperture 209 is blocked and images are projected onto the retina by the electronic projection systems 135; and a third mode in which vision is provided only through the passthrough aperture 209 and in which the electronic projection systems 135 do not project images onto the retina. In some embodiments, the ocular device 125 includes two different instances of the structure 279, one instance being the electronic layer that is adjustable between transparent and opaque, and the other instance being the polarized lens. In this manner, the user may selectively block the light path 211 using either method, e.g., putting the polarized layer 281 in front of the eye (e.g., by donning glasses that include the polarized layer 281) or by providing user input that causes the control circuitry to adjust the electronic layer to be opaque.

In accordance with aspects of the present disclosure, the images that are projected onto the retina by the electronic projection systems 135 may correspond to real world images received at the electronic imaging systems 130 to provide a baseline of standard vision to the user. In various embodiments, the projected images may be enhanced using one or more of selective magnification of areas or objects in the images, thermal imaging, brightness and contrast control, color saturation/desaturation, or image sharpening. These enhancements may be achieved using one or more image processing techniques carried out by control circuitry 240 in the ocular device 125 or by an external computer device that communicates wirelessly with the ocular device 125 via the wireless communication system 225.

In accordance with some aspects of the present disclosure, the ocular device 125 is configured to function as a virtual reality (VR) device in the second mode of vision in which the passthrough aperture 209 is blocked and images are projected onto the retina by the electronic projection systems 135. In a VR mode, the ocular device 125 may communicate with an external computer device for generating the images that are projected onto the retina by the electronic projection systems 135.

In accordance with aspects of the present disclosure, the ocular device 125 is configured to function as an augmented reality (AR) device in the first or second modes of vision described above. In one example of an AR mode of the ocular device 125, real-world images seen through the passthrough aperture 209 are augmented using images projected onto the retina by the electronic projection systems 135. In another example of an AR mode of the ocular device 125, the optical path 211 through the passthrough aperture 209 is blocked, and real-world images captured by the electronic imaging systems 130 are augmented electronically by the ocular device 125 or an external computer device, and these augmented images are projected onto the retina by the electronic projection systems 135. In embodiments, such augmentation may include but is not limited to adding closed-captioning, adding descriptive text, adding user-defined visual information, and other information specific to the incoming visual information (e.g., real world images) received from the front-facing electronic imaging systems 130.

In accordance with some aspects of the present disclosure, the ocular device 125 is configured to be controlled by user inputs comprising predefined voice input, predefined eye movements, predefined face movements, or predefined hand movements. In embodiments, the user inputs are detected by an external computer device that communicates wirelessly with the ocular device 125 via the wireless communication system 225. This enables the user to choose between the various display modes described above, e.g., standard vision, enhanced vision, virtual reality, or augmented reality.

In various embodiments, the ocular device 125 is specifically configured via its shape and size to be implanted in a human eye. In this regard, in the ocular device 125 sub-circuit chips of the electronics may be thinned using wafer thinning techniques to be thin enough such that the entire system is such that the thickness dimension D2 satisfies the expression 1 mm<=D2<=3 mm. These techniques are employed in stacked memory chips with wafers thinned to less than 20 μm thick and bonded to other wafers and connecting micro-TSVs are made between active layers that are 10 μm to 20 μm tall. The ocular device 125 may be constructed such that a length dimension D1 satisfies the expression 1 mm<=D1<=10 mm and a width dimension D3 satisfies the expression 1 mm<=D3<=5 mm. An ocular device 125 having these dimensions D1, D2, and D3 is suitable for implanting an eye (e.g., a human eye), such as shown in FIGS. 1 and 6. In a non-limiting example, the ocular device 125 has dimensions D1=7 mm, D2=2.5 mm, and D3=4 mm.

In accordance with aspects of the present disclosure, the images that are projected onto the retina by the respective projection systems 135 correspond to the incoming light received at the respective imaging systems 130, such that the ocular device 125 may be used as an artificial vision system that can be used to treat vision disorders such as blindness. By using at least two imaging systems 130 and projection systems 135, e.g., one imaging system and projection system pair for frontal vision and one imaging system and projection system pair for peripheral vision, embodiments may be used to provide near perfect vision to the user.

In various embodiments, and as shown in FIG. 4, the structure 207 comprises one or more walls that define an opening and the passthrough aperture 209 and the imaging systems 130.1 and 130.2 are arranged within a perimeter of the opening when viewed from a front of the ocular device 125. This is to provide an unobstructed path for light from outside the eye to enter the passthrough aperture 209 and the imaging systems 130. As further shown in FIG. 4, in some embodiments the passthrough aperture 209 is closer to the center of the opening defined by the structure 207 compared to the imaging systems 130.1 and 130.2 that are further away from the center. In one example, a diameter 299 of an opening (e.g., circle) defined by the structure 207 in this view is about 3-5 mm.

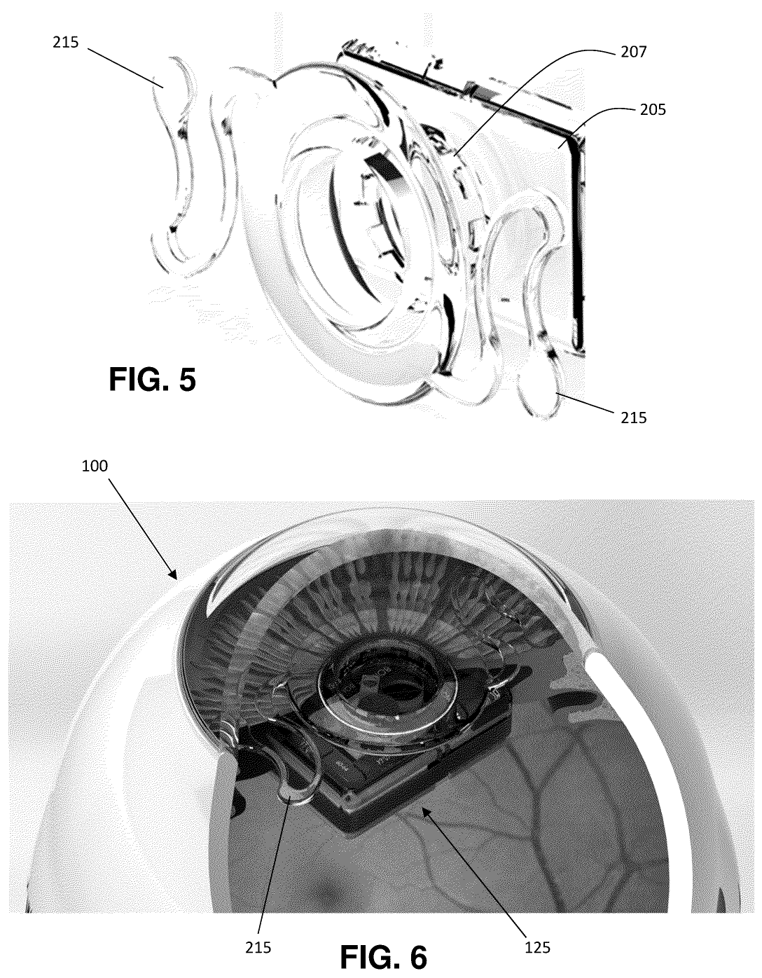

FIG. 5 shows an exemplary implementation of the haptics 215, structure 207, and body 205 in accordance with aspects of the present disclosure. In this example, the body 205 comprises a case that houses the electronics of the ocular device 125, a first end of the structure 207 (i.e. the pupil collar) is connected to the case, and the haptics are connected to a second end of the structure 207 opposite the first end of the structure. As shown in FIG. 5, the structure 207 may be shaped like a cylinder and may optionally have holes in the wall of the cylinder.

FIG. 6 shows an exemplary implementation of the ocular device 125 implanted in an eye 100 of a user in accordance with aspects of the present disclosure. As shown in FIG. 6, the haptics 215 are forward of the iris, the central body is rearward of the iris, and the iris sits in the seat formed by the central body, the structure (i.e. the pupil collar), and the central body.

FIG. 7 illustrates wireless charging in accordance with aspects of the present disclosure. In various embodiments, an external device comprising eyeglasses 705 includes a wireless charging system 730 that is configured to wirelessly charge the power source 235 of the ocular device 125. In one example, the wireless charging system 730 comprises a charging coil that cooperates with the charging coil 220 in the ocular device 125 to perform inductive charging of the power source 235 of the ocular device 125. The eyeglasses 705 may include its own power source, such as an on-board battery or wired connection to an external power source, that powers the wireless charging of the power source 235 of the ocular device 125. In embodiments in which the power source 235 does not store an appreciable amount of power or is omitted, the wireless charging system 730 is configured to wirelessly provide power to the ocular device 125 for operating the electronics during the time that the power is wirelessly provided.

In one example, the eyeglasses 705 comprise eyeglasses and the lens of the eyeglasses comprises the polarized layer 281 shown in FIG. 3. In this manner, when the user dons the eyeglasses 705 to charge the ocular device 125, the light path 211 is blocked and the ocular device 125 uses the second mode of vision described herein.

FIG. 8 diagrammatically shows an exemplary optimization of relative locations of the passthrough aperture 209 and the imaging systems 130.1 and 130.2 in accordance with aspects of the present disclosure. FIG. 8 shows a diagrammatic view of the location of the optical device 125 relative to the nose 805 of the user when the optical device 125 is implanted in the eye of the user. In embodiments, the optical device 125 is structured so that the passthrough aperture 209 is closer to the nose 805 than are the imaging systems 130.1 and 130.2, when the optical device 125 is implanted in the eye of the user. In this manner, light entering the wide-angle imaging system 130.2 is as less likely to be obstructed by the nose 805.

Various embodiments include methods of using an artificial vision system in accordance with aspects of the present disclosure. Various embodiments include methods of implanting an artificial vision system in accordance with aspects of the present disclosure. Such methods may include implanting the ocular device into the eye of the user.

In accordance with aspects of the present disclosure, and as described herein, various embodiments provide enhanced or simulated vision using an electronic device such as ocular device 125 that includes dimmable (e.g., selectively blocked or unblocked) centrally wide-angle passthrough aperture and multiple optimized focal lenses (associated with multiple imaging systems and projection systems) to optimize to the eye's natural vision. Implementations of this device may be used as a therapeutic intra-ocular device that shifts images digitally to preferential areas of the retina that are not affected by geographical atrophy, such as with age-related macular degeneration, for example.

An exemplary embodiment uses multiple imaging systems 130 on the front of the ocular device 125 and multiple projection systems 135 on the back of the ocular device 125, where each of the systems has a specialized optical lens which enable different focal characteristics for the different systems. These systems and their associated lenses are located off-center from the centrally located passthrough aperture 209 and passthrough lens. This method and configuration minimizes power and pixels and maximizes therapeutic efficacy. In this embodiment, the passthrough aperture 209 includes a polarizing lens behind it for selectively blocking and unblocking the optical path 211 through the passthrough aperture 209. This central lens provides wide-angle passthrough vision when the charging eyeglasses 705 are not worn.

In this embodiment, this projected visual data into the visual field occurs when the optical path 211 is blocked, e.g., due to the electronic layer that can be dimmed (e.g., made opaque) or by using an external polarized layer 281 in combination with an internal polarized lens. Such an embodiment can provide 99% or more of visual light attenuation though when the charging eyeglasses 705 including the polarized layer 281 are worn by the user. The eyeglasses 705 may also include blinders such that the only light through the charging glasses comes through the imaging systems of the ocular device 125.

In accordance with some aspects of the present disclosure, one purpose of the passthrough aperture 209 with wide-angle lens is to maximize vision including providing peripheral vision to the user when the ocular device 125 is turned off or unpowered. For example, there may be situations when none of the imaging systems 135 are projecting images onto the retina. In such situations, the passthrough aperture 209 with wide-angle lens provides the user with natural vision.

In accordance with some aspects of the present disclosure, when the eyeglasses 705 including the polarized layer 281 are worn by the user, the optical path 211 through the passthrough aperture 209 is blocked. However, when the eyeglasses 705 are off, the optical path 211 is clear and the user still has their normal un-aided vision including peripheral vision. Having peripheral vision when the ocular device 125 is turned off or unpowered is advantageous so that the user does not bump into objects which can result in injury. Loss of peripheral vision is a known problem issue with some conventional therapeutic IOL devices. For example, the IMT (implantable miniature telescope) is a therapeutic IOL device that effectively eliminates the user's peripheral vision in the affected eye requiring extensive physical therapy. Polarized film approaches for blocking the optical path, as described herein, have the advantage of not being able to be stuck in an “off” mode as in the case of an electronic light-valve, which represents an advantage from a safety point of view.

In accordance with some aspects of the present disclosure, the ocular device 125 when turned on also projects central and peripheral vision onto the retina through multiple specialized imaging and projection systems that are configured to project images onto the area from the center of the retina extending out to 60 or more degrees off-retina center. This is advantageous for addressing AMD where the user's retina may not be very functional within 10 degrees off center of the retina. In embodiments described herein, the multiple imaging and projection systems with different optical lenses can be used to optimize vision improvement on a per user basis, and not just with respect to pixel/power optimization, but in terms of peripheral vision and power/pixel minimization while maximizing visual field and reducing obstructions.

As described herein, the ocular device 125 may constitute an electronic device that has a passthrough aperture 209 defining an optical path 211 that can be selectively blocked and unblocked electronically and/or using polarization elements, and that also projects information onto the back of the retina using a power-optimized (e.g., low power) system comprising multiple imaging systems and multiple projection systems.

As described herein, the ocular device 125 enables as method of optimizing vision improvement to a user with geographic atrophy that allows shifting magnified/improved vision onto still-functioning portions of the retina while optimally matching to the projection of a passthrough aperture 209 defining an optical path 211 that can be selectively blocked and unblocked electronically and/or using polarization elements.

As described herein, the ocular device 125 may constitute an electronic device that has a passthrough aperture 209 defining an optical path 211 that can be selectively blocked and unblocked electronically and/or using polarization elements, and that provides vision enhancements such as selective magnification, thermal imaging, brightness and contrast control, color saturation/desaturation, and image sharpening.

As described herein, the ocular device 125 may constitute an electronic device that has a passthrough aperture 209 defining an optical path 211 that can be selectively blocked and unblocked electronically and/or using polarization elements, and that can function as a virtual reality device by communicating through an external computer device.

As described herein, the ocular device 125 may constitute an electronic device that has a passthrough aperture 209 defining an optical path 211 that can be selectively blocked and unblocked electronically and/or using polarization elements, and that can function as an augmented reality device by adding functionality to vision such as closed-captioning, descriptive text, user-defined visual information, and other information specific to the incoming visual information from the front-facing camera.

As described herein, the ocular device 125 may constitute an electronic device that has a passthrough aperture 209 defining an optical path 211 that can be selectively blocked and unblocked electronically and/or using polarization elements, and that can be by the user using voice control, predefined eye movements, predefined face movements, or predefined hand movements. In embodiments, this enables the user to choose between the various display modes described above, e.g., standard vision, enhanced vision, virtual reality, or augmented reality.

As described herein, the ocular device 125 may constitute an electronic device that has a passthrough aperture 209 defining an optical path 211 that can be selectively blocked and unblocked electronically and/or using polarization elements, and that is configured to use artificial intelligence (AI) to identify objects (e.g., people) in the visual field and then then selectively move (e.g., eccentrically fixate) or magnify the identified object based on user preferences. In embodiments, the ocular device 125 may communicate wirelessly with an external computer device that runs or accesses an AI program for object identification in images. Based on identifying an object in an image from one of the imaging systems 130, the object may be enhanced (e.g., using image processing such as selectively moved or magnified), and that image may be projected onto the retina by one or more of the projection systems.

As described herein, the ocular device 125 may constitute an electronic device that has a passthrough aperture 209 defining an optical path 211 that can be selectively blocked and unblocked electronically and/or using polarization elements, and that includes a pupil collar (e.g., structure 207) that is configured to prevent the iris from interfering with the optical path 211.

As described herein, the ocular device 125 may constitute an electronic device that has a passthrough aperture 209 defining an optical path 211 that can be selectively blocked and unblocked electronically and/or using polarization elements, and that collects images using the imaging systems and uses information generated in the ocular device to project images onto the retina using the projection systems.

In various embodiments, at least of the multiple imaging system of the ocular device 125 is configured to capture images of the retina rather than collecting light from outside the eye. For example, one of the imaging systems may include a structure that causes light from inside the eye to impinge upon a lens and image sensor. The respective structures may comprise one or more reflective surfaces and/or prisms that define a circuitous optical path, e.g., as described in U.S. application Ser. No. 19/195,971. In this manner, one or more of the imaging systems may be used to collect light from inside the eye in which the ocular device is implanted, which may be used in mapping healthy and unhealthy (e.g., damaged) portions of the retina of the eye in which the ocular device is implanted, which may be used to determine regions onto which to project images as described herein. These images of the retina captured by the ocular device 125 may be analyzed using image analysis techniques to differentiate between healthy regions and damaged regions of the retina. The image analysis may be performed by the control circuitry 240 of the ocular device 125, or may be performed by an external computing device (e.g., smartphone, tablet computer, laptop computer, desktop computer, server, etc.) that communicates with the ocular device 125, e.g., via the wireless communication antenna 225.

In embodiments, the ocular device 125 stores data that is based on a mapping of the retina 105 for a particular eye of a particular user and that is used to control the operation of the first projection system 135.1 and the second projection system 135.2. The mapping of the retina upon which this data is based may include a definition of the first region 145.1, the second region 145.2, and the damaged region. Using such data, the ocular device 125 may be configured to not project light onto a damaged region of the retina. One method of doing this is to selectively turn off (e.g., not use) individual light emitting elements (e.g., LED pixels) in one or more of the projection systems that are mapped to the damaged region. By selectively not using individual light emitting elements in this manner, the power consumption of the ocular device 125 is reduced compared to when all the light emitting elements are used to project. The mapping may be determined in the manner described in US Patent Application Publication No. 2025/0099299, the contents of which are incorporated by reference herein in their entirety. In another example, the mapping may additionally or alternatively be determined by capturing images of the retina in which the ocular device is implanted, e.g., by using at least of the multiple imaging system of the ocular device 125 to capture images of the retina rather than collecting light from outside the eye.

As described herein, the ocular device 125 may constitute an electronic device that has a passthrough aperture 209 defining an optical path 211 that can be selectively blocked and unblocked electronically and/or using polarization elements, and that is configured to digitize the visual field to provide improved vision to people suffering from macular degeneration and other forms of eye damage. By digitizing vision, there are numerous additional functionalities that may be provided to people to enhance their experience. In some of these cases, this is accomplished by projecting data, in the form of images, text or symbols, colors, brightness, and positional information, onto the retina that does not correspond to 1:1 magnification with an unenhanced visual field. The data may be generated by the ocular device 125 via local image or data processing in the control circuitry 240, or the data may be provided to the ocular device 125 via wired or wireless digital communication with an external computer device. The external computer device may include but is not limited to virtual reality devices, personal computers, cell phones, brain interface devices, or devices sharing web-based information.

As described herein, the ocular device 125 may constitute an electronic device that has a passthrough aperture 209 defining an optical path 211 that can be selectively blocked and unblocked electronically and/or using polarization elements, and that is configured to enhance vision compared to a user's natural vision in many ways. For example, the ocular device 125 can be fit with a variety of camera lenses to provide a wide range of focal lengths, including the equivalent of a wide-angle or a zoom lens. Projecting the visual information from these lenses would artificially magnify (or demagnify) one's vision relative to natural vision. In another example of enhancing a user's vision relative to their natural vision, the ocular device 125 can further enhance the magnification with digital zoom, i.e., projecting only a subset of the camera image onto the entirety of the projected image. This magnification may be performed in multiple ways including: (1) set in a clinical setting and applied to the same area(s) indiscriminate of the objects in the visual field; (2) set by the user using an app or similar personal interface device and applied to either the same area(s) indiscriminate of the objects in the visual field or using onboard image detection to apply the magnification to people, faces, animals, or other filters; or (3) using onboard artificial intelligence combined with onboard image detection to selectively magnify objects that the user would want. In another example of enhancing a user's vision relative to their natural vision, the ocular device 125 can further enhance vision with specific respect to the blind spot or distorted region of a user with AMD by selectively translating a subset of the full collected image to a projected image.

In various embodiments of the ocular device 125, digital wide-angle lenses are used to gather and project peripheral vision in relatively low-resolution minimizing pixels (ideally: +/−45 degrees). A second set of narrow field lens, which may comprise folded/prism lenses, provided forward for higher resolution field of view input and projection.

In various embodiments of the ocular device 125, the pupil collar (e.g., the structure 207) beneficially minimizes the chance that the iris will constrict and thus block the optical path 211 defined by the a passthrough aperture 209. In various embodiments of the ocular device 125, the imaging systems 130.1 and 130.2 are located apart from the passthrough aperture 209 in the space defined by the pupil collar to avoid obstruction, e.g., as shown in FIG. 4. On the front side of the ocular device 125, the imaging systems 130.1 and 130.2 and the passthrough aperture 209 are all arranged in the space defined by the pupil collar, e.g., as shown in FIG. 4, to avoid the iris obstructing light entering the passthrough aperture 209 or imaging systems 130; however, on the back side of the ocular device 125 the projection systems are not limited to locations defined by the pupil collar and can be located outside of that area.

In some embodiments, the ocular device 125 comprises a contact lens that is configured to be placed on an exterior of the eye or an implantable contact lens that is configured to be implanted in the eye. In these embodiments, the passthrough aperture, the electronic imaging systems, the electronic projection systems, and the other electronics are packaged in a contact lens shaped form factor. In these embodiments, the passthrough aperture, the electronic imaging systems, the electronic projection systems, and the other electronics function in a similar manner as described herein. These components may have different sizes, shapes, and locations in a contact lens compared to an the device shown in FIG. 4, for example, due to the different form factors of contact lenses.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An electronic device configured to be implanted in or on an eye, the electronic device comprising:
a passthrough aperture defining an optical path extending through the electronic device from a front side of the electronic device to a back side of the electronic device;
multiple imaging systems configured to receive incoming light at the eye; and
multiple projection systems configured to project images onto multiple different regions of a retina of the eye based on the incoming light received by the imaging systems.

2. The electronic device of claim 1, wherein the different regions of the retina include:
a first region associated with frontal vision; and
a second region associated with peripheral vision.

3. The electronic device of claim 2, wherein the projection systems comprise:
a first projection system configured to project first images onto the first region at a first resolution; and
a second projection system configured to project second images onto the second region at a second resolution that is lower than the first resolution.

4. The electronic device of claim 1, wherein the projection systems are configured to simultaneously project respective different images onto respective ones of the different regions of the retina.

5. The electronic device of claim 1, further comprising an electronic layer in the optical path, wherein the electronic layer is adjustable between a first state in which the electronic layer is transparent and a second state in which the electronic layer is opaque and blocks the optical path.

6. The electronic device of claim 1, further comprising a polarized lens in the optical path.

7. The electronic device of claim 6, further comprising an external device comprising a polarized layer having a first orientation of polarization, wherein the polarized lens has a second orientation of polarization opposite the first orientation of polarization such that the polarized layer and the polarized lens combine to block the optical path when the external device is placed in front of the electronic device.

8. The electronic device of claim 7, wherein the polarized layer is optically transparent to the imaging systems when the external device is placed in front of the electronic device.

9. The electronic device of claim 1, wherein the electronic device comprises a central body and haptics.

10. The electronic device of claim 9, wherein the passthrough aperture, the imaging systems, and the projection systems are contained in a chip or chip stack, or attached to a printed circuit board, in the central body.

11. The electronic device of claim 9, further comprising control circuitry, a power source, a wireless communication system, and a wireless charging system.

12. The electronic device of claim 9, further comprising a structure extending between the central body and haptics.

13. The electronic device of claim 12, wherein the structure, the central body, and the haptics define a seat that is configured to receive an iris of the eye when the electronic device is implanted in the eye.

14. The electronic device of claim 13, wherein when the electronic device is implanted in the eye the haptics are configured to be in an anterior chamber of the eye forward of the iris and the central body is configured to be behind the iris.

15. A method comprising implanting the electronic device of claim 13 in the eye of a user such that the haptics are in the anterior chamber of the eye forward of the iris and the central body is behind the iris.

16. The electronic device of claim 13, wherein:
the structure comprises one or more walls that define an opening; and
the passthrough aperture and the imaging systems are arranged within a perimeter of the opening when viewed from a front of the electronic device.

17. The electronic device of claim 13, wherein:
the central body comprises a case having that encapsulates a chip or chip stack containing the passthrough aperture, the imaging systems, and the projection systems;
a first end of the structure is connected to the case; and
the haptics are connected to a second end of the structure opposite the first end of the structure.

18. The electronic device of claim 1, wherein the electronic device configured to provide vision enhancement comprising one of selective magnification of areas or objects, thermal imaging, brightness and contrast control, color saturation/desaturation, or image sharpening.

19. The electronic device of claim 1, wherein the electronic device is configured to operate as a virtual reality device.

20. The electronic device of claim 1, wherein the electronic device is configured to operate as an augmented reality device.

21. The electronic device of claim 1, wherein the electronic device is configured to be controlled using voice control, predefined eye movements, predefined face movements, or predefined hand movements.

22. The electronic device of claim 1, wherein the electronic device comprises one of: a contact lens that is configured to be placed on an exterior of the eye; or an implantable contact lens that is configured to be implanted in the eye.
